(19) United States
(12) Patent Application Publication
(10) Pub. No.: US 2011/0245633 A1
(43) Pub. Date: Oct. 6, 2011

(54) DEVICES AND METHODS FOR TREATING PSYCHOLOGICAL DISORDERS

(75) Inventors: Robert Goldberg, Lincoln, MA (US); Shailendra Yadav, Lexington, MA (US)

(73) Assignee: Neumitra LLC, Cambridge, MA (US)

(21) Appl. No.: 13/040,816

(22) Filed: Mar. 4, 2011

Related U.S. Application Data
(60) Provisional application No. 61/310,280, filed on Mar. 4, 2010.

(51) Int. Cl. A61B 5/0205; A61B 5/024; A61B 5/0402; A61B 5/08; A61B 5/01; A61B 5/053; A61B 5/0488; A61B 5/0476; A61B 5/021; A61B 5/103; A61B 5/1455; A61B 5/0496

(52) U.S. Cl. 600/301; 600/508; 600/523; 600/529; 600/549; 600/547; 600/546; 600/544; 600/485; 600/587; 600/323

(57) ABSTRACT

A wearable biosensor device gathers physiological data from the wearer and uses this information over time to diagnose, detect, monitor, and treat psychological disorders. The device triggers real-time psychological treatments based on personalized estimates of the wearer stored on the biosensor device. A therapeutic stimulus is selected from a library based on the data received from the wearable biosensor device and relating to psychological condition(s), and that stimulus is delivered to the wearer via an associated display. The aggregate data from use of the device is provided to clinicians and/or patients, in detail and in summary report forms, to indicate the symptoms of, detect and diagnose among disorders or subtypes, to analyze treatment effects, and to isolate the underlying causes of one or more psychological disorders or states.

### CROSS-REFERENCE TO RELATED APPLICATION

This claims priority to and the benefit of Provisional U.S. Patent Application Ser. No. 61/310,280, filed Mar. 4, 2010, the entire contents of which are herein incorporated by reference.

### TECHNICAL FIELD

The invention generally relates to wearable devices, and systems and methods for detecting, diagnosing, monitoring, and treating a psychological condition based on physiological parameters specific to the wearer and detected by the device.

### BACKGROUND INFORMATION

Treatments for psychological disorders require on-going evaluation by a trained mental health professional. Few laboratory tests are available for psychological disorders, however, and this makes diagnosis, monitoring, and treatment of psychological disorders difficult and time consuming. Clinicians must base their evaluations on limited patient contact, relying on a patient's self-reported experiences, behavior indicated by relatives and/or friends, and various mental health examinations. A diagnosis is arrived at by comparing this information to that in the Diagnostic and Statistical Manual of Mental Disorders, commonly referred to as the DSM-IV (IV for fourth edition, 1994), which uses a system devised by the American Psychiatric Association to classify psychological disorders. An appropriate therapeutic regimen is then selected based on the diagnosis. For treatment the clinician must rely on the patient's accuracy and depth of self-reported experiences to assess the efficacy of any given therapeutic regimen, making on-going evaluations more problematic. Clinicians currently lack the ability, in any robust way, to monitor a patient's progress in-between follow-up appointments, making it difficult to assess treatment efficacy and adjust the regimen as necessary with symptomatic changes. Whereas other branches of medicine use objective data to evaluate the health status of patients, treatments for psychological disorders have so far been driven by subjective reports.

Moreover, epidemiological data indicates that the majority of those with psychological disorders do not seek-out or follow through with psychological or psychiatric care due to the associated social stigma of treatments, including time consuming visits to mental health practitioners and the medications available are accompanied by significant side effects. As such, psychological disorders for the vast majority of patients are undiagnosed, misdiagnosed, mistreated, and/or untreated, resulting in a higher incidence of crime and suicide-related deaths, increased treatment costs, and lower quality of life. For example, hundreds of thousands of military soldiers and veterans suffer from post-traumatic traumatic stress disorder (PTSD) which oftentimes goes undiagnosed and/or untreated due to a reluctance to seek treatment by the many soldiers/veterans and/or the lack of capacity/resources to fully treat PTSD at a majority of VA hospitals across the nation. As such, the number of suicide-related deaths and domestic abuse incidences among soldiers and veterans has dramatically increased over the past two decades. The military currently lacks the ability to continuously monitor the stress level of for each of its soldiers returning from combat or pre-deployment, or to identify those suffering from or at-risk of PTSD who may pose a risk of harm to themselves or their loved ones. These difficulties in delivering adequate mental health treatments are also prevalent in society-at-large. For instance, of the estimated 40 million Americans with anxiety-based disorders each year, less than 10 million will seek treatment, and fewer than 2 million will find adequate treatment. Without novel approaches to mental health treatments, the public will continue to struggle with finding sufficient care.

### SUMMARY OF THE INVENTION

The invention provides devices and methods for monitoring one or more physiological parameters of a subject on a round-the-clock basis, and for using accumulated physiological data (i.e., objective symptom metrics) to affect psychological/psychiatric treatments in real-time and over the long-term. In particular, the invention provides a wearable biosensor device for continuously measuring one or more physiological parameters associated with symptoms of a psychological disorder, and a system that implements the accumulated information regarding the physiological changes detected by the wearable biosensor device to deliver just-in-time therapeutic stimuli to the user/wearer of the device. Over the longer-term, this psychologically-relevant physiological data is brought to bear on treatment decisions. For clinicians and patients, such accumulated data profiles are used to adjust medication dosing, increase medication compliance, adjust treatment strategies, and demonstrate therapy effectiveness. Prospectively, these data profiles are used to identify among individuals at-risk for psychological disorders, such as traumatic experiences like combat and natural disasters, as well as detect and diagnose among particular types and subtypes of psychological disorders.

The wearable biosensor device contains an on-board processor that is configured to derive a psychological profile based on the physiological data detected by the sensor and accumulated over time. The accumulated and derived data is stored in a local data file on the wearable device to create a personalized profile unique to the individual wearer. The personalized profile is regularly and/or continuously updated based on ongoing monitoring with the wearable biosensor device, including the wearer's response patterns to previous treatment/therapeutic stimuli and their on-going response to new stimuli. The wearable sensor device checks a detected physiological state against the personalized profile to determine when to present an appropriate real-time therapeutic stimulus, such as cognitive behavioral therapy, exposure therapy, and/or relaxation techniques. As such, the invention provides devices and methods that enable “personalized medicine” for mental health treatments on-demand and driven by the wearer's current mental state.

Not only is the therapeutic stimulus selected based on an individual's specific physiological and/or psychological state measured at a given point in time (and over their continuum), but the therapeutic stimulus itself can be pre-selected by the individual wearing the device so as to have a maximal psychological and/or emotional impact specific to the given individual. Moreover, the wearable biosensor device and therapeutic delivery system are wireless and discrete, thereby lending themselves to increased patient compliance over the long-term. Such long-term use enables robust treatment analyses supported by data-driven dashboards and reports to highlight the wearer's symptom profile, response to particular treatments, including medications and therapies, and their overall mental health. These reports are automated to allow for efficient, but extensive, symptom reviews, within individual wearers and across large groups of current or potential patients, such as military units, clinical drug trials, and/or research studies.

In one aspect, the invention provides a wearable biosensor device that includes at least one sensor for measuring physiological data, memory for storing the accumulated physiological data over time, an on-board processor for deriving a psychological profile based upon said accumulated physiological data, and an interface for displaying information concerning said psychological profile. The derived data, encompassing physiological and subjective states, is packaged and pushed and/or pulled to remote processors as necessary to produce long-term dashboards and reports to inform and/or alert the wearer and their caregivers to treatment trends and results. These remote processors are also used as necessary to add clinical treatment data to the wearer's symptom profile.

The psychological profile is unique to an individual wearer and can represent a psychological state characterized by a plurality of physiological data/parameters, including but not limited to heart rate, pulse rate, beat-to-beat heart rate variability, electrocardiography (ECG), respiration rate, skin temperature, core body temperature, heat flow off the body, galvanic skin response (GSR), electromyography (EMG), electroencephalography (EEG), electrooculography (EOG), blood pressure, hydration level, muscle pressure, activity level, body position, or a combination thereof. Subjective data, reported by the patient, and treatment data, reported by the clinician, is combined with these physiological measurements to produce a cumulative data profiles tied to the individual wearer.

The information displayed to the wearer on the interface of the biosensor device, or on associated/connected devices, can be an alert of an impending symptomatic event, a diagnosis based on the psychological profile derived from the physiological data, a questionnaire for the user regarding his current mental state or activity, instructions to the user (e.g., to “take a deep breath”, or “relax”), a visual stimulus (e.g., an image of a calming scene, a picture of a loved one, an amusing video, an inspiration phrase or quote), or any combination thereof. The information collected from these displays are tied to the physiological data in the wearer's stored profile.

The wearable device can further include a transmitter for conveying the psychological profile or detected/accumulated physiological data directly to an associated electronic device such as a mobile phone, a smart phone, a digital personal assistant, a laptop computer, a tablet, an e-reader, a desktop computer, a television, a gaming device, or a remote server. Preferably, the transmitter wirelessly transmits the data to the electronic device in real-time. Suitable wireless transmitter systems include but are not limited to an IrDA, a Bluetooth™, a UWB, a Z-Wave, ANT, RFID, or a ZigBee transmitter system/network. The wearer's profile data can be transferred through these means to enable personalized displays on any of the associated electronic devices, stored profiles for initializing new sensors, or profiles to initialize new treatment providers. This transmission of the accumulated data can include to supporting clinicians, caregivers, family members and other individuals or institutions affiliated with the wearer to provide oversight and treatment responses.

In another aspect, the invention provides an all-in-one, self-contained, wearable biosensor device for detecting, diagnosing, monitoring and treating a psychological disorder and/or a psychological state in a subject that includes at least one sensor for detecting one or more physiological parameters (e.g., heart rate, pulse rate, beat-to-beat heart rate variability, electrocardiography (ECG), respiration rate, skin temperature, core body temperature, heat flow off the body, galvanic skin response (GSR), electromyography (EMG), electroencephalography (EEG), electrooculography (EOG), blood pressure, hydration level, muscle pressure, activity level, body position, or a combination thereof), a digital media library, a processor configured for deriving data indicative of a psychological disorder and/or psychological state (e.g., an anxiety disorder, post-traumatic stress disorder, obsessive-compulsive disorder, panic disorder, a phobic disorder, depression, bipolar disorder, a psychotic disorder, addiction, autism, attention deficit hyperactivity disorder, schizophrenia, stroke recovery, traumatic brain injury, an eating disorder (e.g., anorexia nervosa, bulimia nervosa, binge/compulsive over-eating, purging, etc.), chronic pain/pain management, a baseline state, etc.) based on the detected physiological parameter and selecting a media from the library based on the derived data, a display for presenting the selected stimulus to a subject, and memory for storing accumulated data detected by the biosensor and derived by the processor.

The digital media library can include audio files, video files, text files, still images, questionnaires, or any combination thereof, and can be a personalized media selection, selected by the individual wearing the biosensor device and/or selected by supporting clinicians and/or caregivers. Deliveries from this library can be driven by the physiological parameters and the individual's psychological profile, or some combination thereof, and based on real-time events, treatment plans, the wearer's preferences, and/or automated based on the patterns seen in the physiological data or psychological profiles and/or wearer demographics.

The on-board processor includes one or more algorithms for recognizing patterns in the detected physiological parameters accumulated over time. Suitable pattern recognition algorithms include machine learning algorithms such as Dynamic Baysian Networks, neural networks, conditional random fields, hidden Markov models, Kalman filters, fuzzy logic, kernel estimations, k-nearest neighbor, learning vector quantization, Gaussian models, and/or radial basis function. These patterns can be derived from calibrating events, in a caregiver's presence or on their own, as presented on the associated displays, and used to indicate individual differences, generalized response profiles as from disorder types, or wearer demographics. Such patterns can also be derived on the associated devices and tied to those displays.

The all-in-one, self-contained, wearable biosensor device can further include a transmitter for sending the data detected by sensor and/or derived by the on-board processor directly to an electronic device such as a mobile phone, a smart phone, a digital personal assistant, a laptop computer, a tablet, an e-reader, a desktop computer, a television, a gaming device, or a remote server. Preferably, the transmitter wirelessly transmits the data to the electronic device in real-time or in packets accumulated over time. Suitable wireless transmitter systems include but are not limited to an IrDA, a Bluetooth™, a UWB, a Z-Wave, ANT, RFID, or a ZigBee transmitter system/network.

In yet another aspect, the invention provides a system for detecting, diagnosing, monitoring and treating a psychological disorder and/or psychological state. The system includes a wearable biosensor device that includes at least one sensor for detecting one or more physiological parameters (e.g., heart rate, pulse rate, beat-to-beat heart rate variability, electrocardiography (ECG), respiration rate, skin temperature, core body temperature, heat flow off the body, galvanic skin response (GSR), electromyography (EMG), electroencephalography (EEG), electrooculography (EOG), blood pressure, hydration level, muscle pressure, activity level, body position, or a combination thereof), a processor configured for deriving data indicative of a psychological state (e.g., anxiety, panic, depression, mania, a substance-abuse related craving, a baseline state, etc.) based on said detected physiological parameter, memory for storing accumulated data detected by said sensor or derived by said processor, and a transmitter for wirelessly sending data directly to an electronic device for display on the electronic device upon receipt of the transmitted data by the electronic device. The system further includes the electronic device which has a display and contains a digital media library. The electronic device is configured to select and play a media from said library on said display based on data received from the wearable biosensor device.

The electronic device may be a smart phone, a digital personal assistant, a laptop computer, a tablet, an e-reader, a television, a gaming device, or a desktop computer.

The digital media library can include audio files, video files, text files, still images, questionnaires, or any combination thereof, and can be a personalized media selection selected by a given individual using the system.

The processor on-board the wearable biosensor component of the system includes one or more algorithms for recognizing variations and patterns in the detected physiological parameters accumulated over time. Variations are indicated from the individual's baseline or from sample- or population-level estimates. Suitable pattern recognition algorithms include machine learning algorithms such as Dynamic Baysian Networks, neural networks, conditional random fields, hidden Markov models, Kalman filters, fuzzy logic, kernel estimations, k-nearest neighbor, learning vector quantization, Gaussian models, and/or radial basis function.

The transmitter included on-board the wearable biosensor component of the system transmits data directly to an electronic device such as a mobile phone, a smart phone, a digital personal assistant, a laptop computer, a tablet, an e-reader, a desktop computer or a remote server. Preferably, the transmitter wirelessly transmits the data to the electronic device in real-time. Suitable wireless transmitter systems include but are not limited to an IrDA, a Bluetooth™ a UWB, a Z-Wave, ANT, RFID, or a ZigBee transmitter system/network.

The invention further provides methods for detecting, diagnosing, monitoring and treating one or more psychological disorders and/or psychological states, including but not limited to anxiety disorders, post-traumatic stress disorder, obsessive-compulsive disorder, panic disorder, phobic disorders, depression, bipolar disorder, a psychotic disorder, and addiction, autism, attention deficit hyperactivity disorder, schizophrenia, stroke recovery, traumatic brain injury, eating disorders (e.g., anorexia nervosa, bulimia nervosa, binge/compulsive over-eating, purging, etc.) and pain management.

In one aspect of the methods of the invention, an all-in-one, self-contained, wearable biosensor device is provided to an individual for detecting, diagnosing, monitoring and/or treating a psychological disorder and/or psychological state. The wearable biosensor device includes at least one sensor for detecting one or more physiological parameters (e.g., heart rate, pulse rate, beat-to-beat heart rate variability, electrocardiography (ECG), respiration rate, skin temperature, core body temperature, heat flow off the body, galvanic skin response (GSR), electromyography (EMG), electroencephalography (EEG), electrooculography (EOG), blood pressure, hydration level, muscle pressure, activity level, body position, or a combination thereof), a processor configured for deriving data indicative of a psychological state (e.g., anxiety, panic, depression, mania, a substance-abuse related craving, or a baseline state) based on the detected physiological parameter, a digital media library, on-board memory, and a display. The current psychological state of the user is determined using the wearable biosensor device. The current psychological state is compared against a local data file and/or data storage in which accumulated physiological data, or a summarized profile thereof, has been stored in on-board memory over time, to arrive at the current psychological state. An appropriate therapeutic stimulus is selected from the digital library based on the derived psychological state and presented to the individual wearing the device on the display.

The on-board digital media library can include audio files, video files, text files, still images, questionnaires, or any combination thereof, and can be a personalized media selection selected by a given individual using the system.

The processor on-board the wearable biosensor device includes one or more algorithms for recognizing variations and patterns in the detected physiological parameters accumulated over time. Variations are indicated from the individual's baseline or from sample- or population-level estimates. Suitable pattern recognition algorithms include machine learning algorithms such as Dynamic Baysian Networks, neural networks, conditional random fields, hidden Markov models, Kalman filters, fuzzy logic, kernel estimations, k-nearest neighbor, learning vector quantization, Gaussian models, and/or radial basis function.

The on-board memory in which the local data file is stored has the capacity to store extensive data, for example, at least 12+ hours of data, preferably more (e.g., 1000+ hours of data), and can be in the form of a memory chip, card or stick. Preferably, the memory is flash memory, and can be expandable as necessary.

The wearable biosensor device can further include a transmitter for sending the accumulated and/or derived data directly to an electronic device such as a mobile phone, a smart phone, a digital personal assistant, a laptop computer, a tablet, an e-reader, a desktop computer, a television, a gaming device, or a remote server. Preferably, the transmitter wirelessly transmits the data to the electronic device in real-time. Suitable wireless transmitter systems include but are not limited to an IrDA, a Bluetooth™, a UWB, a Z-Wave, ANT, RFID, or a ZigBee transmitter system/network.

In another aspect of the methods of the invention, a system including a wearable biosensor device and an associated electronic device is provided to an individual for diagnosing, detecting, monitoring and treating a psychological disorder and/or psychological state. The wearable biosensor device includes at least one sensor for detecting one or more physiological parameters (e.g., heart rate, pulse rate, beat-to-beat heart rate variability, electrocardiography (ECG), respiration rate, skin temperature, core body temperature, heat flow off the body, galvanic skin response (GSR), electromyography (EMG), electroencephalography (EEG), electrooculography (EOG), blood pressure, hydration level, muscle pressure, activity level, body position, or a combination thereof), a processor configured for deriving data indicative of a psychological state (e.g., anxiety, panic, depression, mania, a substance-abuse related craving, or a baseline state) based on the detected physiological parameters, on-board memory, and a transmitter for sending accumulated and/or derived data directly to an electronic device such as a mobile phone, a smart phone, a digital personal assistant, a laptop computer, a tablet, an e-reader, a desktop computer, a television, a gaming device, or a remote server. The electronic device includes a display and a digital media library.

The wearable biosensor device is used to measure one or more physiological parameters and the current psychological state of the user is determined using the wearable biosensor device. The current psychological state is compared against a local data file in which accumulated physiological data has been stored in the on-board memory over time, or a summarized profile thereof, to arrive at the current psychological state. The wearable biosensor then transmits data to the electronic device regarding the current psychological state, including a set of instructions regarding an appropriate media to select from the digital library based on the derived psychological state, and the selected media is presented to the individual wearing the device on the electronic device.

The digital media library stored on the electronic device component can include audio files, video files, text files, still images, questionnaires, or any combination thereof, and can be a personalized media selection selected by a given individual using the system.

The processor on-board the wearable biosensor device component includes one or more algorithms for recognizing variations and patterns in the detected physiological parameters accumulated over time. Variations are indicated from the individual's baseline or from sample- or population-level estimates. Suitable pattern recognition algorithms include machine learning algorithms such as Dynamic Baysian Networks, neural networks, conditional random fields, hidden Markov models, Kalman filters, fuzzy logic, kernel estimations, k-nearest neighbor, learning vector quantization, Gaussian models, and/or radial basis function.

The on-board memory in which the local data file is stored on-board the wearable biosensor device has the capacity to store at least 12 hours of data, preferably more (e.g., 1000+ hours of data), and can be in the form of a memory chip, card or stick. Preferably, the memory is flash memory, and is expandable as necessary.

In all aspects of the devices, systems and methods of the invention, the wearable biosensor devices include one or more sensors, such as a galvanic skin response (GSR) sensor, a temperature sensor, a heart rate sensor, an oxygen saturation sensor, a blood pressure sensor, or a combination thereof. Preferably, the wearable biosensor devices at least include a GSR sensor. Optionally, the wearable devices can further include an accelerometer and/or a global positioning system (GPS). The wearable biosensor devices can even further include a clock, and a button for time-stamping events/daily activities by a subject wearing the biosensor device.

In all aspects of the devices, systems and methods of the invention, the wearable biosensor devices include a power source for providing power to at least the sensor, the memory and the processor, such as a silver, alkaline, mercury, zinc-air or lithium button, coin or watch cell.

In all aspects of the devices, systems and methods of the invention, the wearable biosensor devices can include an LED display, such as a multi-colored LED display, for signaling or alerting the wearer of a detected physiological and/or psychological condition or state (e.g., red LED=extremely stressed/anxious/agitated; yellow LED=warning, anxiety/agitation level rising; rising; green=normal/relaxed/baseline state).

In all aspects of the devices, systems and methods of the invention, the wearable biosensor devices are preferably adapted for wearing around a wrist (e.g., watch, a bracelet), an ankle (e.g., an ankle cuff), a finger (e.g., a ring), a torso, an arm (e.g., an arm band), a leg (e.g., a leg band), or a foot (e.g., a sock or a shoe).

In all aspects of the devices, systems and methods of the invention, the on-board memory of the wearable biosensor devices has the capacity to store at least 12 hours of data, preferably more (e.g., 1000+ hours of data), and can be in the form of a memory chip, card or stick. Preferably, the memory is flash memory, and is expandable as necessary.

The wearable biosensor devices, systems, and methods of the invention can be used by clinicians and health professional to help monitor patients both in and out of the clinician's office, and thus can be used to diagnose and treat psychological disorders. Additionally, round-the-clock monitoring using the personalized wearable biosensor devices of the invention will better inform clinicians and patients about how to manage and treat a given psychological disorder and/or psychological state. For example, the wearable biosensor devices of the invention are useful in helping a patient identify factors that trigger a psychological episode, and helps a patient recognize when they are experiencing a psychological episode based on physiological factors associated with the episode. The associated physiological factors detected by the wearable sensor device (which may be specific to the wearer), cues the immediate delivery of a therapeutic stimulus to the wearer of the device to alleviate the episode. The aggregate data from use of the device is provided to clinicians and/or patients, in detail and in summary report forms, to indicate the symptoms of, to monitor and analyze treatment effects, to detect and diagnose among disorders or subtypes, and to isolate the underlying causes of one or more psychological disorders and/or states. This aggregate data can be displayed over any of the associated devices and using secure protocols to protect the wearer's privacy.

The wearable biosensor devices, systems, and methods of the invention are also useful in helping to predict the onset of a psychological episode and can prevent the episode by delivering a therapeutic stimulus to the wearer of the device coincident with the onset of symptoms. The delivery of said therapeutic stimulus can arrive in the forms of a visual, auditory, or tactile alert cuing the wearer to an impending or on-going symptomatic event. By tracking long-term trends associated with the use of the wearable biosensor, the biosensor device enables treatment analyses associated with the onset and offset of medications and clinical treatment decisions, indicates patient relapses associated with the reoccurrence of symptoms, and highlights symptom trends in a personal profile stored on-board the biosensor device and transferable to the associated devices.

The wearable biosensor devices, systems, and methods described herein are particularly useful for round-the-clock monitoring of subjects suffering from an anxiety disorder such as PTSD, panic disorder, and social phobia; obsessive-compulsive disorder; specific phobias such as agoraphobia and glossophobia; as well as subjects suffering from anxiety disorders, post-traumatic stress disorder, obsessive-compulsive disorder, panic disorder, phobic disorders, depression, bipolar disorder, a psychotic disorder, and addiction, attention deficit hyperactivity disorder, stroke recovery, traumatic brain injury, autism, schizophrenia, sleep disorders, chronic pain, and eating disorders (e.g., anorexia nervosa, bulimia nervosa, binge/compulsive over-eating, purging, etc.). The devices and systems described herein further provide real-time therapeutic intervention or prevention of symptomatic episodes related to such disorders. With increasing wear, the biosensor becomes highly attuned to the variance of physiological symptoms (variance from a normal/relaxed/baseline state) tied to the individual wearer and the treatment course becomes increasing personalized to the individual.

Various aspects, features, objects, advantages, and details of the invention herein disclosed will become apparent through reference to the following description, the accompanying drawings, and the claims.

### BRIEF DESCRIPTION OF THE DRAWINGS

In the drawings, like structures and items typically are referenced by the same or similar reference numbers throughout the various views. The illustrations in the drawings are not necessarily drawn to scale, the emphasis instead being placed generally on illustrating the principles of the invention and the disclosed embodiments.

FIG. 1 is a schematic depicting an exemplary embodiment of a wearable bio sensor device according to the invention for configured for wearing on the wrist or ankle.

FIG. 2 is a flowchart depicting exemplary data transmission pathways according to exemplary methods of the invention.

FIG. 3 shows exemplary embodiments of an Annotate Panel, an Activity Panel for patient self-reporting, and a therapeutic stimulus, that can be displayed on a wearable sensor device or associated electronic device.

FIG. 4 is a flowchart depicting an exemplary embodiment of a data processing path in the wearable sensor devices of the invention.

FIG. 5 is a flow chart depicting an exemplary embodiment of real-time monitoring and treatment methods according to the invention.

FIG. 6 is a schematic depicting a wellness loop provided by the devices and methods of the invention.

FIG. 7 is a flow diagram of a centralized computing infrastructure and dashboard in an exemplary embodiment of the invention.

### DESCRIPTION

The invention provides devices, systems, and methods for continuous monitoring of one or more physiological parameters of a subject (such as clinical patient or soldier) and indicating and/or treating psychological disorders. In particular, the invention provides wearable biosensor devices and systems for detecting one or more physiological parameters in the subject wearing the device, correlating the detected physiological parameter with a particular psychological state, and delivering a therapeutic stimulus based on the detected physiological/psychological state to the subject in real-time. With increasing use, the wearable biosensor becomes highly-specific to the individual wearer for rapid detection of symptomatic episodes and personalized treatments are delivered as necessary. This personalization is built into the sensor and associated methods, with a wearer profile stored on the device and/or associated electronic devices, and accessed during regular use. The personalized functioning of the biosensor may be transferred to any other device but remains specific to the wearer. This specific profile of the wearer determines the type and timing of stimulus presentation on the wearable device and/or on associated electronic devices for the purpose of therapeutic treatments. The aggregate data from the use of the device, and specific to the wearable, is applied in diagnosis, detection, and monitoring of one or more psychological disorders and/or psychological states on the wearable biosensor devices and/or on associated electronic devices.

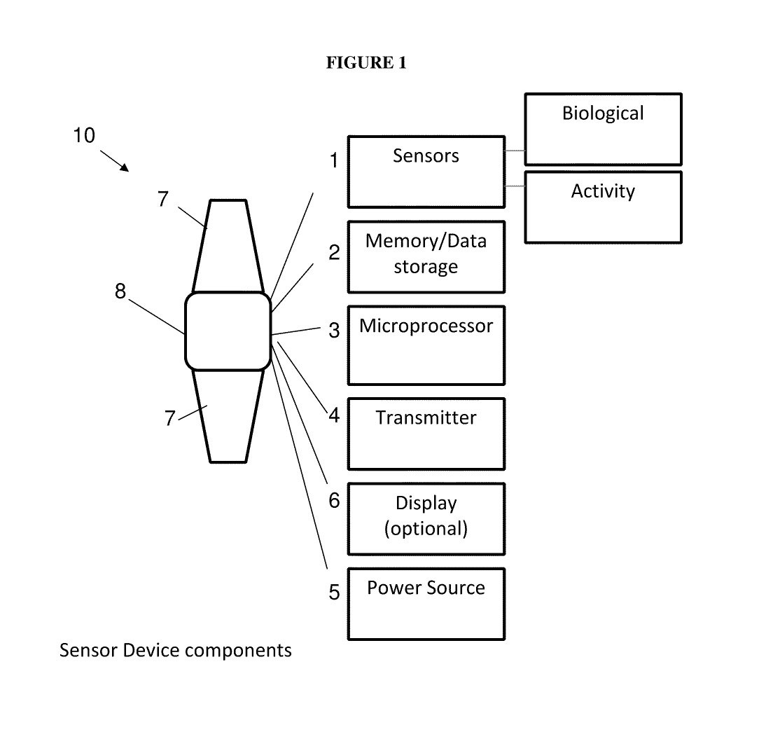

An exemplary embodiment of the wearable biosensor device of the invention is depicted in FIG. 1. As shown in FIG. 1, the wearable bio sensor device 10 of the invention includes one or more sensors 1 for measuring one or more physiological parameters and/or activity level, memory/data storage capacity 2, a processor or microprocessor 3 for reading/analyzing the physiological data detected by the one or more sensors, a transmitter 4 (preferably a wireless transmitter), a power source 5 (e.g., a battery), and an optional display 6. The sensors 1, memory 2, processor 3, transmitter 4, power source 5 and optional display 6 are mounted or encased within a central housing 8 and attached to a wearable component 7. The embodiment depicted in FIG. 1 is a modular design includes a band that can be comfortably worn around or attached to the body, such as on the wrist (e.g., bracelet or watch form), an ankle (an ankle cuff), a finger (e.g., a ring form), a torso, an arm (e.g., an arm band or cuff), a leg (e.g., a leg band or cuff), a foot (e.g., a sock or a shoe form).

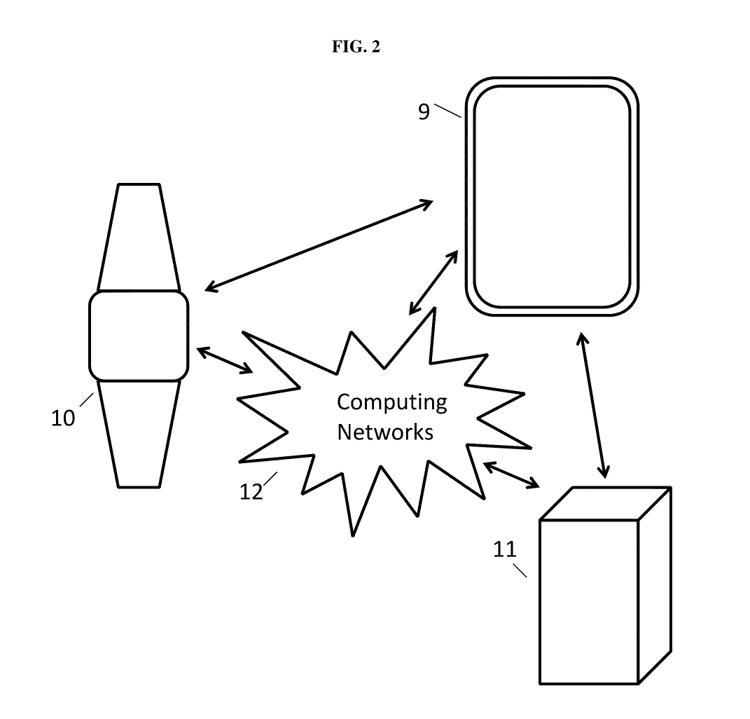

FIG. 2 is a flow chart that depicts a exemplary embodiments of various data transmission pathways in accordance with methods of the invention. In the embodiment shown in FIG. 2, a user wears a battery-powered biosensor device 10 for measuring one or more physiological parameters. The wearable biosensor device 10 contains a processor configured for analyzing and deriving data indicative of a psychological state based on the physiological data collected by the biosensor. The processed data is continually stored on a local file in the wearable biosensor device. The processor analyzes the detected physiological data in real-time based on a personalized calibration file (information specific to the wearer) that is stored on the device. The wearable biosensor device then transmits the detected and/or derived data over a personal area network to an electronic device 9 such as a mobile phone, a smart phone, a digital personal assistant, a personal laptop computer, a desktop computer, a tablet, a television, a gaming device, or an e-reader. The electronic device 9 contains a digital media library containing s audio, visual, text, and video stimuli that serves as therapeutic stimuli for the treatment of psychological disorders and/or psychological states. Upon receiving the transmitted data from the wearable biosensor device 10, the electronic device presents 9 (e.g., via a display screen and/or a speaker system and/or an actuator) a selected media from the digital library to the individual wearing the biosensor device 10. The media is selected based on the data received from the wearable biosensor device 10. Alternatively, the digital library can be contained on-board the wearable biosensor device, such that the wearable biosensor device is an all-in-one monitoring and treatment system capable of detecting a physiological parameter, deriving data indicative of a psychological state based on the detected physiological parameter and using a highly personalized profile of the wearer, selecting a therapeutic stimulus from the digital media library, and presenting the selected therapeutic stimulus to the wearer of the device.

An alternative embodiment of a method according to the invention is depicted in FIG. 2, in which the wearable biosensor device 10 interfaces with electronic device 9 and/or centralized computing infrastructure 11, as described above, via a cloud computing network 12 (virtual computation, software, data access, and storage services that do not require end-user knowledge of the physical location and configuration of the system that delivers the services). In such a configuration, the information received from the wearable biosensor can be accessed by the patient, their family or caregivers, and supervising clinicians for the purposes of remote diagnosis, detection, monitoring and tracking of symptom profiles specific to the wearer.

In many applications, it is desirable for the sensors to operate on a long-term, round-the-clock basis. As such, the wearable biosensor devices must be comfortably worn for long periods of time (days and weeks) by adults and/or children without interfering with daily activities, such as sleeping, washing hands, or typing. Additionally, it is desirable for the sensors to be worn in discrete locations in order to increase patient compliance, particularly among members of the military, police force, fire fighters, and other high risk and/or high-stress occupations. As such, it is desirable that the wearable biosensor devices be in a comfortable, discrete, washable form factor, such as an armband, a wristband, a bracelet or watch-like device, a hand band or glove, a finger ring, an ankle band, a shoe, or a sock.

The material which forms the wearable band in which the one or more sensors are included, or to which the one or more sensors are attached, is preferably made of a comfortable, flexible, breathable material. In certain embodiments, a flexible, breathable, hydrophobic material is used such as Gore-Tex® (sold by W. L. Gore & Assoc., Newark, Del.), or Dryline® (sold by Milliken & Company, Spartanburg, S.C.). This stretchable fabric is hydrophilic on the inner layer and hydrophobic on the outer layer, so that moisture moves away from the wearer's skin through the fabric to the outer layer, where it evaporates. Alternatively, other hydrophobic, breathable materials may be used. For example, eVent® fabric (sold by BHA Group, Inc., Kansas City, Mo.) or Epic® fabric (sold by Nextec Applications, Inc., Bonsall, Calif.) may be utilized. In some embodiments, a synthetic stretch mesh, such as 85% nylon and 15% Lycra® may be used. Fabrics comprising a mix of elastic and leather may also be used to advantage.

In certain embodiments, a flexible closure is used fasten the two ends of the wearable band together. For instance, the flexible closure may include Velcro® strips or a metal fastener.

The wearable biosensor devices may contain one or more sensors for gathering physiological data regarding heart rate (sympathetic and parasympathetic arousal), pulse rate, beat-to-beat heart rate variability, electrocardiography (EKG or ECG), respiration rate, skin temperature, core body temperature, heat flow off the body, galvanic skin response (GSR), electromyography (EMG), electroencephalography (EEG), electrooculography (EOG), blood pressure, hydration level, muscle pressure, activity level, body position, and/or optical reflectance of blood vessels.

In a particular embodiment, the wearable biosensor devices of the invention at least include one or more sensors that measures electrodermal activity (EDA), also known as galvanic skin response (GSR), which measures sympathetic arousal. The electrodes for EDA sensors can be made of one or more electro-conductive materials, including conductive fabrics and yarns, conductive polymers, conductive elastomers or metal. In a particular embodiment, the EDA sensors are metal electrodes, such as silver-silver chloride electrodes, that are mounted or partially encased within a housing, with the electrodes exposed to allow contact with a skin surface. The housing in which the electrodes are mounted or partially encased can be attached to a wearable fabric band that can be worn, for example, around the arm, wrist, or ankle. Alternatively, the metal electrodes may be detachably mounted on a wearable fabric band using pop-in snaps or the like. Metal snaps may be used to connect the electrodes (or leads from them) to the circuit (or lead from it). When the snaps are snapped together, the electrodes and circuitry are electrically connected; when they are snapped apart, they are not electrically connected. These snaps thus enable the circuitry to be repeatedly attached to and detached from the wearable band with electrodes. The wearable band with electrodes can then be easily washed or replaced. The placement of the metal snaps may vary. For example, the snaps may be near the electrodes, or near the circuitry instead. Alternatively, other electrical connectors may be used instead of the metal snaps. In some implementations, the electrical connector is light-weight and at least one part of the connector is washable.

The EDA sensors can also be made of a medical-grade silver-plated 92% Nylon 8% Dorlastan® fabric (Cat. #A251, Less EMF, Inc., Albany, N.Y.). This electro-conductive fabric is washable, allows the skin to breathe, maintains elasticity and provides consistent contact with the skin. Alternatively, the electrode can be made of electro-conductive thread or yarn embroidered into fabric or other material. For example, a stainless steel electro-conductive thread sold by Bekaert (Winston Salem, N.C.) can be used. This enables greater comfort and durability since the conductive thread exhibits less strain fatigue than traditional metal wires. Alternatively, electrically conducting elastomers or polymers may be used for the electrodes. Poly(3,4-ethylenedioxythiophene), also known as PEDOT, is an example of such a conducting elastomer. Carbon-impregnated rubber is an example of such a conducting polymer. These conductive elastomers and polymers are not generally breathable and thus less desirable. This problem may be solved in some cases by aeration (i.e., hole-punching) that makes the material more breathable. For example, carbonized rubber may be aerated in that fashion.

In an exemplary embodiment of the invention, an electrodermal activity (EDA) sensor module implements an exosomatic measurement of EDA, such that a small voltage is applied to the skin and the resulting potential drop is measured. The primary technical challenge in creating this circuit is to achieve a low-power design while still maintaining good dynamic range. It is well known that baseline skin resistance can vary over a few orders of magnitude from 100K Ohms to approximately 10 M Ohms; yet, it is necessary to detect minute changes in this value. Greater dynamic range and sensitivity can be achieved by increasing the voltage rails. Alternately, an EDA sensor circuit may be implemented using a digitally controlled variable gain amplifier to maximize dynamic range. However, this requires the use of an external microcontroller that adds greater cost, complexity, and power consumption.

In some embodiments of this invention, an EDA circuit performs a time-domain measurement of skin conductance by employing an oscillator circuit whose oscillation frequency is dependent on the skin conductance. By measuring this frequency instead of measuring the skin resistance directly, it is possible to perform a more precise measurement given the low power rails and limited dynamic range of the voltage.

In order to maximize battery life and maintain a stable voltage rail for the op-amps and sensors, a low-power low-noise regulator (LM1962, National Semiconductor, Santa Clara, Calif.) may be added. This regulator has a power enable pin that can be used to only momentarily provide power to the biosensor module and power it off when it is not in use, thereby reducing the power consumption of the entire EDA biosensor module to less than 20 microwatts.

The wearable biosensor devices can include one or more additional sensors for measuring a physiological response, in addition to the EDA sensors described above. For example, the wearable biosensor devices can further include a temperature sensor (e.g., a low-power temperature biosensor such as LM60 (National Semiconductor, Santa Clara, Calif.), a heart rate biosensor, an oxygen saturation biosensor, a blood pressure biosensor, or any combination thereof.

In certain embodiments, the wearable biosensor devices include at least one photoplethysmograph (PPG) for measuring heart rate (HR) and heart rate variability (HRV). Since the light absorption of blood is wavelength dependent, if two different wavelength LEDs are used, then it is also possible to measure the relative blood oxygen level using the ratio of readings between the two color LED's.

Conventional PPG devices employ a single LED light. However, this invention may be implemented with a PPG device that has multiple LEDs. In some embodiments of this invention, a PPG photodiode absorbs light reflected from the skin. In other embodiments, a PPG photodiode absorbs light transmitted through tissue.

The wearable biosensor devices can further include a motion sensor. For example, an analog motion sensor (SQ-SEN-200, Signal Quest, Lebanon, N.H.) with an integrator circuit may be used. Advantages of this analog sensor, over an accelerometer, are that it draws less than 1 microamp of current and is inexpensive to purchase. Alternatively, various types of motion sensors may be used, including an accelerometer, such as a 3 axis digital accelerometer. The motion sensor may be any of various types of micro electro-mechanical systems (MEMS) consisting essentially of a proof mass on a damped spring, that measure the deflection of the proof mass in an analog or digital manner. For example, the deflection may be measured by piezoresistors attached to the spring, or by changes in capacitance between fixed beams and beams attached to the proof mass. Also, for example, the accelerometer may have a small heated dome of gas and measure the deflection of the center of the dome.

A motion sensor can also be used to gate the PPG signal so that heart rate data during motion can be ignored or cleaned. It should be noted, however, that there are many times during the day or night when a person's wrists are still, thus allowing for snapshots of HR and HRV. The combination of motion, EDA and HR/HRV are particularly relevant for recognizing sleep stages and conditions such as apnea. In some embodiments, multiple PPG sensors are employed. The multiple PPG signals are combined using signal processing, which reduces noise caused by motion artifacts. In some versions of the invention, logarithmic detection is used, which also helps handle motion artifacts.

This invention may be implemented in such a way that one or more sensors (such as PPG heart rate sensors, motion sensors and temperature sensors) are removable in their entirety from the wearable biosensor. This allows the sensors to be easily removed or replaced, for example, when the band or other host material for the biosensor is washed. In other embodiments, one or more of these sensors are coated in plastic or another waterproof or water-resistant material, so that they can remain with the wrist band (or other wearable garment or material) when it is washed. In the case of PPG sensors, this coating is preferably transparent to the wavelength of light (including red or infrared light) emitted by the LEDs and absorbed by the photodiode. In the case of any temperature biosensor, this coating preferably has a high thermal conductivity. In versions where these sensors remain with a band (or other wearable garment or material) when it is washed, leads may be used to connect the sensors with the removable circuitry, including the radio module and antenna. Metal snaps or other electrical connectors may be used to enable the sensors (or leads from them) to be repeatedly attached to or detached from the removable circuitry (or leads from it).

The wearable biosensor devices may further include a global positioning system to provide information regarding the location of an individual wearing the biosensor device. Such information may be information may be informative of trigger factors or cues that induce or contribute to change in physiological response detected by the one or more sensors in the wearable biosensor device.

The wearable biosensor devices may further include a clock and a button for a user to time-stamp significant events which may induce or contribute to a change in one or more physiological parameters detected by the one or more sensors in the wearable biosensor device.

The wearable biosensor devices of the invention can include an on-board processor that can map patterns of the physiological and motion data to personalized signals or alerts indicative of a likely anxiety attack, panic attack, or other states that the wearer would like to know about, or used to alert other people or devices for assistance, by using, for example, text messages or emails to inform family and clinicians of recent symptomatic events. Preferably, the processor on-board the wearable biosensor device analyzes the physiological data detected by the one or more sensors in real-time using summary metrics and pattern recognition algorithms that become increasingly personalized to the wearer, relying on a personalization profile stored on-board the biosensor device to identify patterns in the data that indicate the need for therapeutic intervention. Alternatively, the pattern analysis and recognition function can be performed in a cloud computing network. In yet another alternative embodiment, pattern analysis and recognition can be performed in a device that directly or indirectly receives data wirelessly from the wearable biosensor device.

In some implementations of this invention, a simple classification scheme that does not involve machine learning may be used to recognize a data pattern. For example, in such a scheme, data may be classified based on criteria derived by simply averaging or aggregating the physiological patterns of multiple users. This scheme may be modified for a particular user's physiology by adjustment-to-baseline and stored as a highly personalized profile file on the wearable biosensor and/or related electronic device (e.g., smart phone, personal digital assistant, laptop computer, tablet, e-reader, television, gaming device, etc.) and integrated into the functioning biosensor device as sensor data is accumulated. For instance, real-time alerts specific to the wearer are increasingly updated and improved based on increasing physiological and/or physical data obtained from the wearer. This personalization profile may be computed on the biosensor itself and/or on a portable electronic device and/or in a networked platform.

In many applications, pattern recognition is more accurate if machine learning is used. For example, machine learning allows a classification algorithm to be customized to take into account differences in affect or context, or cross-user differences in physiology (in a more nuanced manner than merely adjustment-to-baseline). Machine learning algorithms learn from a limited number of examples, where the data may be noisy and contain complex patterns which elude human detection. Expected response functions allow for highly specific modeling of observed data patterns to examine significant effects in the time series data and are tied to the individual wearer in their personalization profile.

Use of a learning machine allows a classification scheme to adapt in response to data. In some embodiments, this gives the processor great flexibility to adjust to complex data patterns that may, for instance, vary within a user over different contexts.

In exemplary embodiments of the invention, machine learning with Dynamic Bayesian Networks (DBNs) is employed to better recognize patterns in physiological, affective, and contextual data. It is advantageous to use DBNs for several reasons. First, DBNs are well-suited for modeling a complex dynamic system. For example, they can be used to model behavioral states confounded by time-varying comorbidities that may come into play in the moments before drug relapse. DBNs are designed to manage noisy data, unknown quantities and uncertain events. A DBN has the power to describe not only instantaneous correlations among variables, but also how their values change over time. Second, DBNs can generalize from limited data because the learning algorithm stresses balancing performance with model complexity. An overly complex model might be able to explain a data set (such as continuous physiology monitoring data) perfectly, but fails to generalize because it is explaining the data's idiosyncrasies (e.g., the humidity that day) of the specific data set. By penalizing model complexity, the algorithm finds the simplest acceptable explanation of the patterns, which are more robust to noise in existing data and tend to generalize better to future data. Third, individual subjects have varying physiology. DBNs are well suited to devising hierarchical models (where data is organized into branching patterns that describe one-to-many relationships) that allow the prediction of physiological changes of an individual person. Fourth, computation in a DBN is efficient: the time required is linear in the length of the sequence and may be performed in real time. Although the complexity of computation does grow with the complexity of the network, the learning algorithm strives to produce a simple network for generalization performance; as a consequence, computation is kept efficient. Thus, a classifier derived from a DBN performs minimal computation to produce an accurate result. This computational efficiency is particularly advantageous if the processor is deployed onboard a mobile device, such as a cell phone.

This invention may be implemented in such a way that a pattern recognition algorithm incorporates prior knowledge (in addition to training data). For instance, prior knowledge may include knowledge of transformation-invariance or knowledge about the data.

In another illustrative embodiment of this invention, a DBN learning algorithm incorporates prior knowledge into a suitable prior distribution over structures, which guides the search toward models that are physiologically relevant while also favoring simple models. Furthermore, the DBN's conditional probability tables (CPT's) are parameterized in a way that incorporates domain-specific knowledge. In an illustrative embodiment, cross-validation is used to set the tunable model parameters. In cross-validation, a portion of the data is withheld from training and instead used for testing; this is repeated across the entire data set.

In some implementations of the invention, the result of the learning algorithm is a structure and parameter set for a DBN. For example, while the training data indicates physiology and context associated with prescription opioid cues, the goal is a classifier to predict State X of relapse risk; this corresponds to using the learned DBN with the relapse status node left unobserved. Prediction of this variable is then made using the Belief Propagation (BP) algorithm, a simple message passing algorithm which operates on the learned network. An advantage of using a DBN is that the computation time required for BP is linear in the length of the sequence, and thus presents no obstacle to implementation in a low-power deployable system.

A learning algorithm can be trained using data to produce a fully specified DBN. The output consists of both the graph structure determining how variables are interrelated, as well as the CPTs that determine how each variable is influenced by its immediate causes in the model. An advantage of using DBNs is that the resulting models are readily interpretable, in contrast to black box approaches such as neural networks.

Alternately, this invention may be implemented with other approaches to machine learning instead of DBNs. For example, it may be implemented with neural networks, conditional random fields, hidden Markov models, Kalman filters, fuzzy logic, kernel estimation, k-nearest neighbor, learning vector quantization, Gaussian models, RBF (radial basis function) classifiers and other statistical classification approaches.

The wearable biosensor devices of the invention further contain on-board memory, thus allowing data collected from the one or more sensors and/or data derived by the processor to be continually stored on the biosensor to influence future biosensor behavior based on the wearer's personal history with the device. The on-board processor and memory capacity eliminates the need for an external server, such as used in other devices and systems described in the art, when comparing real-time data to the stored personalized profile of the wearer. The wearable biosensor can operate in stand-alone mode or in conjunction with an electronic device (e.g., smart phone, personal digital assistant, laptop computer, tablet, e-reader, television, gaming device, etc.) or a remote server. In stand alone mode, the wearable biosensor device is capable of collecting data, processing data, running analytics and delivering therapeutic stimuli without the need of external system. Furthermore, in a stand-alone mode, the sensor stores a local data file (referred to herein as a personalization profile or personalized profile) that becomes unique to the wearer and can be shared across portable electronic devices and networked computing devices. The personalized profile is stored securely locally on the wearable sensor device and is backed-up on associated computing devices. As such, the personalized profile can be loaded onto a new sensor and/or portable electronic device (e.g., a smart phone, personal digital assistant, laptop computer, tablet, e-reader, television, gaming device, etc.) if any previous one is lost or damaged. The adaptive algorithm uses the information in the local personalized profile to adjust presented stimuli to a wearer's specific therapeutic needs in real-time.

Preferably, the on-board memory has the capacity to store several hours to several thousand hours of data, and can be expanded, if necessary. In a particular embodiment, non-volatile computer storage is used, so as to minimize power consumption in the wearable biosensor device. Preferably, flash memory, or some variant thereof, in the form of a memory chip, card, or stick is used in the wearable biosensor devices of the invention.

In addition to the one or more sensors, processor, and memory, the wearable biosensor devices of the invention further include a transmitter for sending data detected by the one or more sensors, and/or data derived by the processor. The transmitter is preferably a short-range wireless transmitter for sending the data directly to an electronic device over a personal area network using a wireless network technology such ANT, IrDA, UWB, Z-Wave, RFID, ZigBee or Bluetooth™. In a particular embodiment, the wearable biosensor device employs Bluetooth™ technology to transmit the data directly to a portable electronic device such as a mobile handheld device (e.g., a cell phone, a smart phone, or a digital personal assistant), a laptop computer, a desktop computer, a tablet or an e-reader, for direct display on the electronic device, without the need for an intermediary hub or radio base station. A microcontroller is included in the wearable biosensor devices for interfacing the Bluetooth™ module, or other data transmission module, with the one or more sensors.

The wearable biosensor devices may optionally contain a user controlled ON/OFF switch or function so the user can choose to turn off the data transmission when desired and/or the same or separate switch for the user to flag events as they occur.

As described above, the one or more sensors in the wearable biosensor devices of the invention detect and monitor one or more physiological parameters, and the on-board processor analyzes the data in real-time and detects/recognizes patterns in the data. In certain embodiments, the on-board processor further includes algorithms for mapping the detected physiological data to a psychological state based on the wearer's personalized profile associated with the device (on-board data file). The on-board processor then generates a set-up of instructions based on the detected and/or derived data. The data and instructions are transmitted, back to the wearable biosensor device (e.g., in an all-in-one monitoring and treatment embodiment), or transmitted, e.g., via a Bluetooth™ network, directly to an associated electronic device, preferably a portable electronic device, as previously described.

In certain aspects of the invention, the wearable biosensor devices can include an LED display, such as a multi-colored LED display. The data and/or instructions generated by the on-board processor are can be translated into an alert or signal to the wearer via the LED display, to alert the wearer in real-time of a detected physiological and/or psychological state or condition (e.g., red LED=extremely stressed/anxious/agitated; yellow LED=warning, anxiety/agitation level rising; rising; green=normal/relaxed/baseline state).

In other certain aspects of the invention, a digital media library is stored in the associated electronic device. The digital media library can contain one or more text files, audio files, video files, still images, or a combination thereof, that serve as therapeutic stimuli to the individual wearing the biosensor device. For example, the digital media library can contain a range of exercises, questionnaires, tests, summary reports, real-time data-driven graphics, audio content (e.g., positive or inspiring quotes, phrases or stories, personal instructions), music content (e.g., classical music, sounds of nature, etc.), video content (e.g., demonstrations of exercises, of calming scenes, etc.) and/or pictures (e.g., of loved ones, favorite scenes, reminders, etc.). Upon receipt of the data and/or instructions from the wearable biosensor device, the electronic device presents, displays or plays a select media file in real-time to the individual wearing the biosensor device (e.g., on a display screen or through speakers contained within the electronic device) based on the personalized profile of the wearer and reflecting previous responses to real-time treatments, thereby providing a therapeutic stimulus (including but not limited to cognitive behavioral therapy, exposure therapy, and breathing techniques such as deep breathing exercises and meditative techniques, photographs, audio, video, and text) to the individual wearing the device in real-time. The selected media is dictated by the data and/or instructions directly received from the wearable biosensor device and is based on the personalized profile of the wearer, reflecting previous responses to real-time treatments.

Alternatively, the digital media library is stored in the on-board memory of the wearable biosensor device, and the therapeutic stimulus is presented to the individual wearing the device (i.e., an all-in-one wearable monitoring and treatment device) based on the personalized profile of the wearer and reflecting previous responses to real-time treatments.

The digital media library can be a pre-selected library of text, audio, video, or image files, based on the individual preferences of the individual wearing the device. In other words, the digital media library can be a personalized selection of media that will have a maximal emotional and/or therapeutic impact on a given individual. The digital media library can also modified as necessary through wearer or clinician actions either on the device itself or remotely through associated devices, such as uploading new media over the internet to the device. One or more media files can be deleted, or uploaded, depending on the preferences of the given individual and/or their clinician.

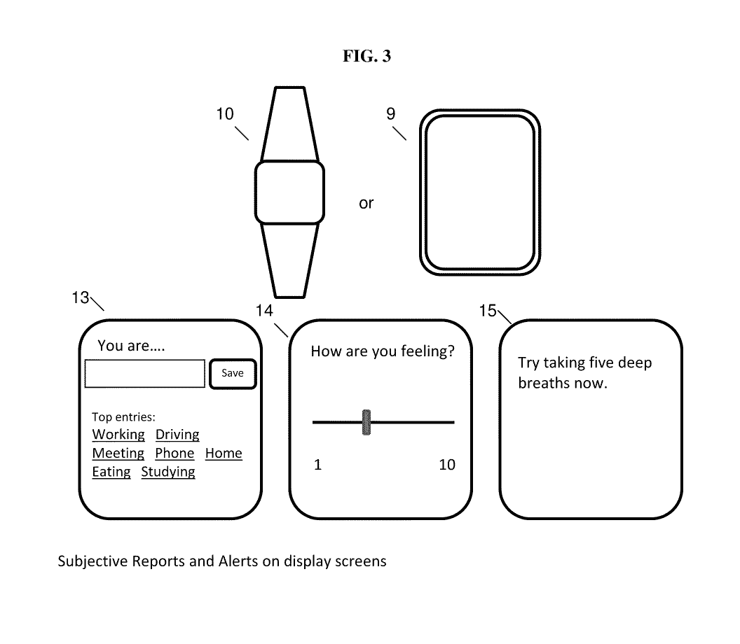

In certain embodiments of the invention, user feedback may be part of the data used to train the data processing algorithm and so the personalization file. This feedback may be obtained in a wide variety of ways. For example, in an application to help a user recognize and/or prevent a psychological episode, a mobile computing device such as a smart phone, a digital personal assistant, a notebook computer, a tablet, television, gaming device, or an e-reader, may display an Annotate Panel and/or an Activity Panel. These panels may be used to gather user feedback, as described below. In the embodiments where the processor is on-board the wearable biosensor device, the gathered user feedback is transmitted back to the wearable biosensor device and/or associated devices to train and correct the algorithm. Alternatively, the wearable biosensor device itself may include and display an Annotate Panel 14 and Activity Panel 13 for gathering user feedback to train the algorithm (FIG. 3). The initial selection of treatments will be further personalized by gathering wearer's resulting physiology on specific stimuli delivered. Over time, the ratings can be used to adjust an adaptive algorithm that will adapt as the wearer's therapeutic outcomes change in response to said stimuli. This adaptive approach enables highly specified physiological and psychological responses of the device and the stimuli tied to the individual wearer.

The Annotate Panel 14 is a graphical user interface (GUI) comprising multiple screens. It allows users to self-report their current mood or mental state (e.g., stress, anxiety, depression, pain exacerbations, frustration, feeling deprived or the need to reward one's self, prescription opioid craving, or any other feeling, behavior, or event they consider interesting). The Annotate Panel also allows a user to self-report his or her response to episode prevention interventions by describing various contexts, events, or situations encountered. Annotations can be completed in any location in which the participant has confidence, and all data is securely stored and transmitted. FIG. 3 shows an example of an Annotate Panel 14 for self-reporting current mood/mental state

An Activity Panel 13 is a GUI that allows a user to self-report his or her current activities, such as when experiencing stress or depression. For example, an Activity Panel may allow a user to select Commute, Working, Personal, Fun, Exercise, Relaxing, Eating, Meeting, Talking or Other, or to input text associated with their experiences. Over time these entries are sorted based on various factors such the most frequent selections, the time of day, and the geospatial location. In this example, the Activity Panel is generally organized with more popular activities at the top of the screen (and therefore easier to identify by the user). Activities most associated with stress and drug craving are placed in easily recognized locations or in separate categories. FIG. 3 shows an example of an Activity Panel 13. In certain embodiments, entering an annotation in an Annotate Panel 14 on an electronic device or on the wearable biosensor device advances the user to an Activity Panel 13, or vice versa.

Preferably, the processor on-board the wearable biosensor device (or alternatively, a cloud computing network) analyzes the physiological data detected by the one or more sensors in real-time, using the personalized profile and/or pattern recognition algorithms to identify patterns in the self-reported data, combined with the collected physiological data, that indicate the need for therapeutic intervention. Therapeutic intervention can be displayed directly on the wearable biosensor device or electronic device in real-time. For example, as shown in FIG. 3, a therapeutic message 15 may be displayed on the wearable biosensor, or on the electronic device instructing the user to “breathe deeply”.

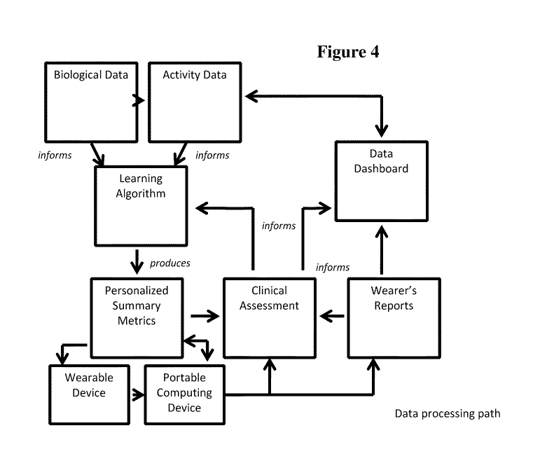

FIG. 4 is a block diagram of high-level functionality the data processing path within a wearable biosensor device that employs a machine learning algorithm, such as a DBN, in an illustrative implementation of this invention. Physiological data is received directly from sensors. In addition, user annotations/activity data can be gathered using an Annotate Panel and Activity Panel on either an electronic device or on the wearable biosensor device. The physiological data, user annotations/activity data, and time of day data, repeated over many samples of these data, make up a set of training data that is used to train a learning algorithm. The learning algorithm produces a personalized profile (denoted in FIG. 4 as “personalized summary metrics”). Prior data can be used to inform the learning algorithm and to verify personalized metrics model. The personalized profile is employed to analyze physiological data in real time, on the wearable device and/or on associated devices, in order to identify patterns, and events and thresholds that indicate the need for therapeutic intervention.

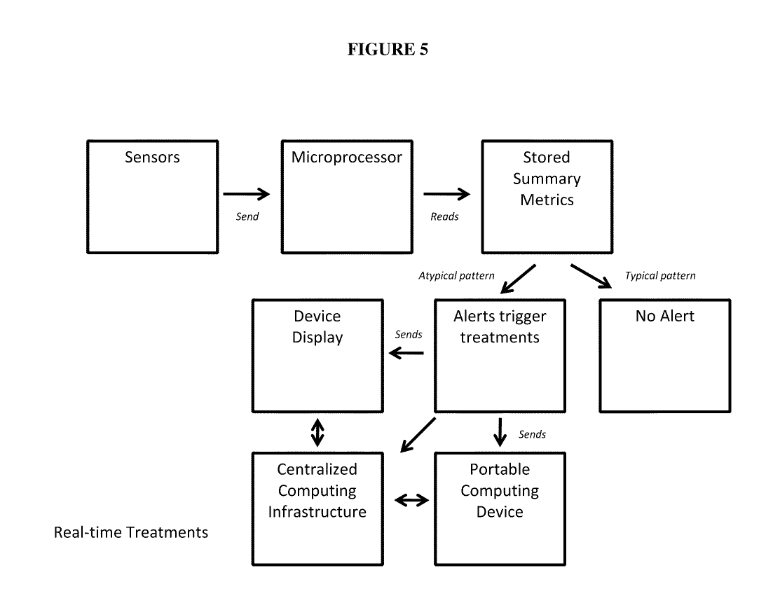

This invention may be implemented as a method comprising the following steps, as shown in FIG. 5. As shown in FIG. 5, physiological/activity data is collected using the wearable biosensor device. A microprocessor on-board the device (or in a cloud computing network) reads/analyzes the data in real-time and sends the data to a local data file for storage and comparison against past data. If an atypical physiological pattern is detected, the wearable biosensor device signals internal logic on the wearable device and/or to an electronic device that triggers real-time delivery of a therapeutic stimuli on the wearable device (i.e. an all-in-one monitoring and treatment embodiment) and/or on an associated electronic device. Alternatively, the therapeutic stimulus can be delivered via the wearable biosensor device itself in an all-in-one monitoring and treatment embodiment. The “alert” can alternatively be transmitted to a centralized computing infrastructure which can store and further process the data or send alerts to caregivers in the form of phone calls, text messages, emails, etc.

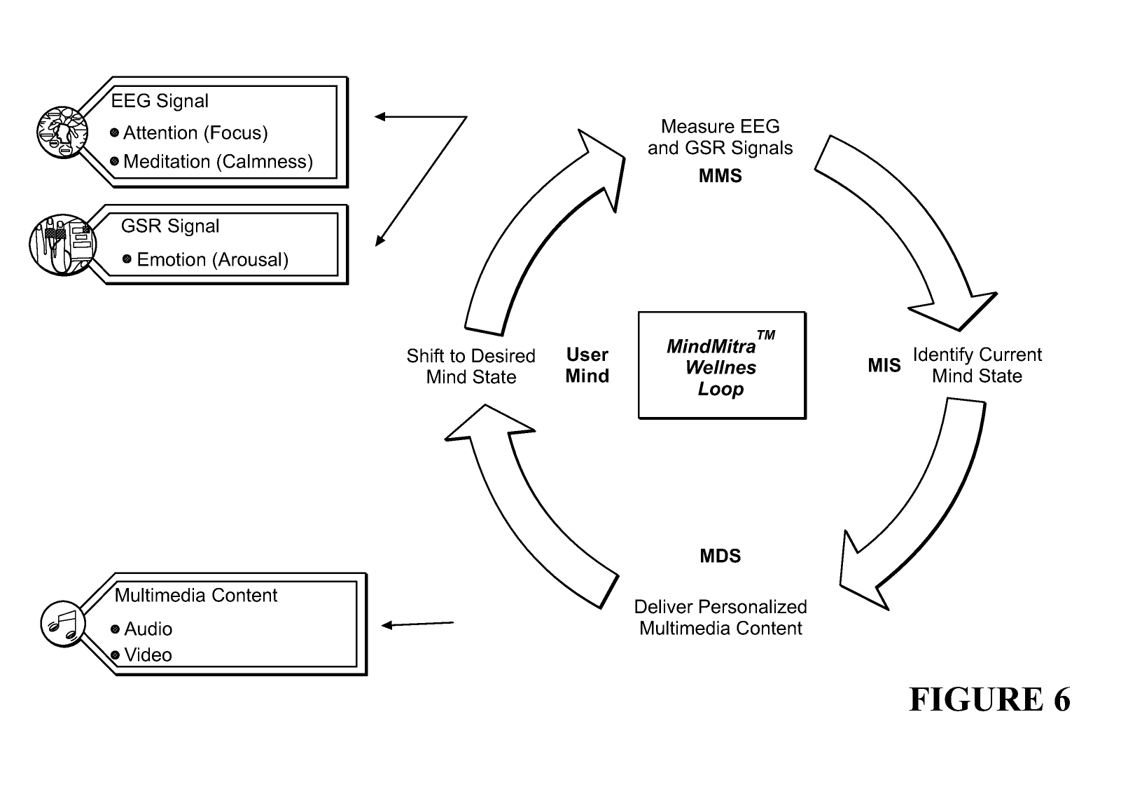

The wearable biosensor devices together with the therapeutic delivery system (contained on-board the wearable device, and/or in a separate electronic device) create a proprietary wellness loop (see FIG. 6) which detects, informs, and improves a given individual's psychological state, or mood on-demand. The loop beings with measuring the user's physiological parameters (biometric signals) in real-time using the wearable biosensor device. The biometric signals are then analyzed by the on-board processor, recorded into the on-board memory, and mapped to a psychological state (e.g., the user's mood) and on the personalized profile of the wearer. A delivery system (e.g., a separate electronic device or the wearable biosensor device itself) uses the information about the reported mental states to deliver personalized information, images, audio or video content to shift the user's current mood based on the physiological data detected an analyzed. The loop timeline will vary depending on the user and mood states. During initial use, the on-board processor learns about the wearer's experience with a specific content (including training protocols) and from the physiological data. Over time, the processor develops an understanding of the user's mood by capturing information on the user's physiology and experiences and storing that updated information in a personalization file tied to the specific wearer that will affect future functioning of the device in the form of real-time stimuli and/or alerts.

The wearable biosensor devices of the invention include a power source to power the one or more sensors, the processor, the wireless transmitter, and microcontroller. Suitable power sources include, for example, button, coin or watch cells, such as a silver, alkaline, mercury, zinc-air or lithium button or cell. In certain embodiments, rechargeable batteries are used to power the sensors, the processor, the wireless transmitter and the microcontroller. This not only eliminates the need to purchase hundreds of batteries that may be needed for long-term use, but enables the battery to be completely embedded inside the wearable device for weatherproofing and safety reasons. Moreover, the biosensor can harness the wearer's motion, thermoregulation, or other events to recharge the battery.

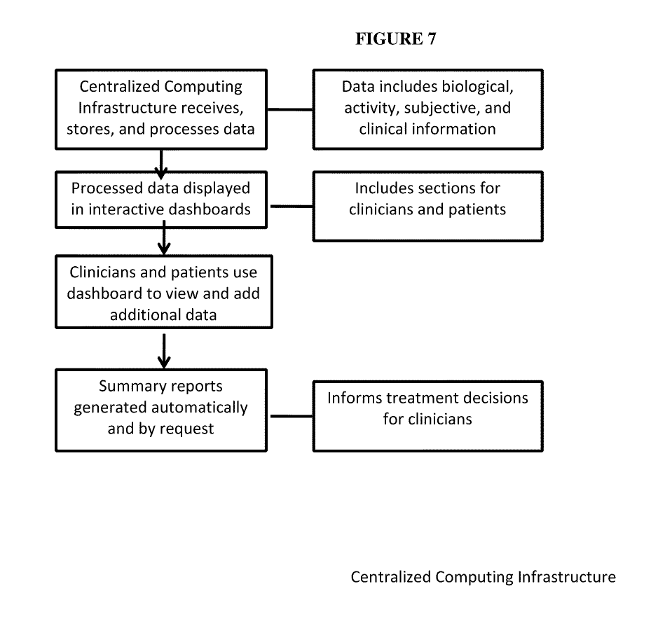

Optionally, the data detected and stored on-board the wearable biosensor devices of the invention is transmitted to a centralized computer infrastructure supporting proprietary data storage and analysis to include clinical summary reports, computed metrics, and correlations with logged activities. For example, data can be wirelessly transmitted from the wearable biosensor device to an electronic device via any number of wireless protocols including, but not limited to Bluetooth™, RFID, cellular, home, and corporate networks. The electronic device then transmits the data, e.g., over a cellular network, or a computer network (e.g., the Internet), to the remote server. Alternatively, the data detected and/or stored on-board the wearable biosensor device can be transmitted to a centralized computing infrastructure via a cellular or a computer network to a third party, such as a clinician or physician, to assist the clinician/physician in diagnosing a psychological disorder and monitoring a patient's progress to inform therapeutic compound dosing schedules and treatment regimens (FIG. 7). Patients and clinicians can access the data stored on the centralized computing infrastructure, for example, via a website, to generate summary reports, or add additional data. The dashboard is used by clinicians and their caregivers, to diagnose psychological disorders, monitor and inform treatment decisions, and can be used to teach patients how to better self-manage their condition. Such embodiments of this dashboard include, but are not limited to, graphs and figures specific to the wearer and updated as new information is available, including, but not limited to, the physiological data, effects of treatment, reports of overall patterns, and self-report information from the Activity and Annotate Panel. This dashboard can be configured for analyses of individual wearers and/or for aggregate reports of groups of wearers such as those found in clinical drug trials or in military units.

The devices and methods described herein have numerous applications. For example, the devices/systems described herein may be implemented such that an individual wears the biosensor device and the sensor/processor/personalized profile detects and recognizes physiological changes in the individual, relative to their normal/baseline physiological state, indicative of a symptomatic episode, such as anxiety or panic. The wearer of the biosensor is alerted of an impending symptomatic episode and is delivered a targeted stimulus, such as a breathing technique, via a display on either the wearable biosensor device or an accompanying portable electronic device, to overcome the anxiety or panic attack.

In another example, an individual has a specific phobia to public speaking. The wearable biosensor device/system can be implemented to alert them to impending changes in their underlying physiology and deliver a therapeutic stimulus (e.g., a soothing song, a motivational/inspiring message, or a reminder to “breathe deeply”) immediately prior to an important business meeting.

In another example, the devices/systems described herein may be implemented such that a soldier/veteran at-risk for PTSD wears the biosensor device/system when returning from a war zone. The sensor/processor/personalized profile on-board the wearable biosensor detects and recognizes physiological changes in the individual relative to their normal/baseline physiological state, indicative of PTSD. The wearable biosensor wirelessly transmits an alert, such as a text message or an email, that indicates to his family and/or his superiors that he should seek treatment from mental health professionals. The devices/systems of the invention can also be utilized by soldiers, police officers, firemen, or other individuals in high-risk/high stress occupations to track their baseline data to reference a healthy mental state prior to experiencing a traumatic event in the line of duty.

In another example, the devices/systems of the invention can be used to diagnose a psychological disorder. For example, an individual reports to mental health professionals with concerns about experiencing on-going depressive episodes. The mental health professional recommends that the individual wear the biosensor device/system around-the-clock each day for a designated time period (e.g., 1 week, 2 weeks, 3 weeks, 4 weeks, 1 month, 3 months, 6 months, 9 months, 1 year, etc.). The physiological data and patterns detected by the wearable biosensor is stored in on-board the personalized profile and/or wirelessly transmitted to a remote server. The data can be downloaded from the biosensor device during a follow-up appointment, or can be accessed by the mental health professional via the dashboard periodically during the designated time period, to assist the mental health professional in distinguishing between major depression, depression with anxiety or depression with aggression, in the individual.

The devices/systems of the invention can also be used to inform a clinician of the efficacy of a therapeutic regimen. For example, a clinician is interested in whether a recently prescribed psychotropic medication is having the desired effect on a patient. The clinician has the patient wear the wearable biosensor device/system around-the-clock each day for a designated time period (e.g., 1 week, 2 weeks, 3 weeks, 4 weeks, 1 month, 3 months, 6 months, 9 months, 1 year, etc.). The physiological data and patterns detected by the wearable biosensor is stored in on-board the personalized profile and/or wirelessly transmitted to a remote server. The data can be downloaded from the biosensor device during a follow-up appointment, or can be accessed by the mental health professional via the dashboard at any point during the designated time period, to assist the mental health professional in determining whether the medication has reduced the patient's symptoms.

The devices/systems of the invention can also be used to inform a patient of the efficacy of a therapeutic regimen. For example, the wearable biosensor device can be advantageously implemented by a psychologist to show a skeptical patient that psychotherapy or medication is gradually reducing their symptoms each week.

The devices/systems of the invention can also be used to inform parents and/or clinicians whether a child has attention deficit hyperactivity disorder. The child wears the wearable biosensor around-the-clock for a designated time period (e.g., 1 week, 2 weeks, 3 weeks, 4 weeks, 1 month, 3 months, 6 months, 9 months, 1 year, etc.). The physiological data and patterns detected by the wearable biosensor is stored in on-board the personalized profile and/or wirelessly transmitted to a remote server. The data can be downloaded from the biosensor device during a follow-up appointment, or can be accessed by parents and/or clinicians via a dashboard at any point during the designated time period. The physiological data and patterns detected by the wearable biosensor is used to examine how the child's emotional state varies throughout the school day.

The devices/systems of the invention can also be used as a deterrent against returning to illegal drug use. For example, a judge orders a criminal defendant on probation to use the wearable biosensor device of the invention. The sensor/processor/personalized profile detects and recognizes physiological changes in the individual, relative to their normal/baseline physiological state, indicative of a drug-craving or drug use. The criminal defendant is alerted of an impending symptomatic episode and is delivered a targeted stimulus, such as a picture of a loved one, via a display on either the wearable bio sensor device or an accompanying portable electronic device, to overcome the drug craving. The dashboard is used indicate the defendant's vigilance to the treatment program.

The devices/systems of the invention can also be used to help athletes overcome athletic difficulties and/or competition anxiety. For example, a professional baseball player experiences difficulty throwing to a base. The wearable biosensor device can be implemented to identify when their anxiety level reaches a peak and to inform how treatment should be approached during training exercises.

The devices/systems of the invention can also be implemented by insurance companies to help plan members track daily stressors and identify mental health risks in an ordinary or at-risk population (e.g., police officers). Aggregate reports are generated to highlight those individuals whose symptom profiles reflect a high likelihood of psychological distress and/or disorder.

The devices/systems of the invention can also be used to inform the efficacy of a clinical drug trial. For example, the wearable biosensor device/system can be used to collect physiological data tied to the drug being tested to provide objective data regarding the physiological effect of the drug and placebo on trial participants.

Certain embodiments according to the invention have been disclosed. These embodiments are illustrative of, and not limiting on, the invention. Other embodiments, as well as various modifications and combinations of the disclosed embodiments, are possible and within the scope of this disclosure.

1. A wearable device for measuring a psychological state, the device comprising:
a sensor for measuring physiological data;
memory for storing accumulated physiological data over time;
a processor for deriving a psychological profile based upon said accumulated physiological data; and
an interface for displaying information concerning said psychological profile.

2. The wearable device of claim 1, wherein said profile is unique to an individual user.

3. The wearable device of claim 1, wherein said physiological parameter is heart rate, pulse rate, beat-to-beat heart rate variability, electrocardiography (ECG), respiration rate, skin temperature, core body temperature, heat flow off the body, galvanic skin response (GSR), electromyography (EMG), electroencephalography (EEG), electrooculography (EOG), blood pressure, hydration level, muscle pressure, activity level, body position, or a combination thereof.

4. The wearable device of claim 1, wherein said psychological profile represents a psychological state characterized by a plurality of physiological data.

5. The wearable device of claim 1, wherein said information is a diagnosis, a questionnaire, instructions to the user, or a visual stimulus.

6. The wearable device of claim 1, further comprising a transmitter for conveying said profile or said information to an electronic device.

7. The wearable device of claim 6, wherein said transmitter wirelessly transmits data to said electronic device in real-time.

8. The wearable device of claim 7, wherein said electronic device is a mobile phone, a smart phone, a personal digital assistant, a laptop computer, a tablet, a television, a gaming device or an e-reader.

9. The wearable device of claim 6, wherein said transmitter is selected from an IrDA, a Bluetooth, a UWB, a Z-Wave, ANT, RFID, or a ZigBee transmitter.

10. The wearable device of claim 1, wherein said sensor is a galvanic skin response (GSR) sensor, a temperature sensor, a heart rate sensor, an oxygen saturation sensor, a blood pressure sensor, or a combination thereof.

11. The wearable device of claim 10, further comprising an accelerometer.

12. The wearable device of claim 11, further comprising a global positioning system.

13. The wearable device of claim 1, wherein said wearable device further comprises a clock.

14. The wearable device of claim 13, wherein said wearable device further comprises a button for time-stamping events by a user wearing said device.

15. The wearable device of claim 1, wherein said memory has capacity to store at least 12 hours of data.

16. The wearable device of claim 1, wherein said memory is a memory chip, card or stick.

17. The wearable device of claim 1, wherein said memory is flash memory.

18. The wearable device of claim 1, further comprising a power source for providing power to at least the sensor, the memory and the processor.

19. The wearable device of claim 1, wherein said wearable sensor device is adapted for wearing around a wrist, an ankle, a finger, a torso, an arm, a leg, a foot.

20. The wearable device of claim 1, wherein said wearable sensor device is configured in the form of a watch, a bracelet, a ring, an arm band, a leg band, an ankle band, a shoe, or a sock.
